(19) United States
(12) Patent Application Publication
(10) Pub. No.: US 2023/0301957 A1
(43) Pub. Date: Sep. 28, 2023

(54) POLYGLYCEROL FATTY ACID ESTER COMPOSITION AND APPLICATION THEREOF

(71) Applicants: SINGAO (XUZHOU) BIOTECH CO., LTD, Xinyi, Jiangsu (CN); SINGAO AGRIBUSINESS DEVELOPMENT CO., LTD., Xiamen City (CN); SINGAO (LONGYAN) BIOTECH CO., LTD., Longyan City (CN)

(72) Inventors: Zhouwen LAI, Xinyi, Jiangsu (CN); Amit Narendra KADAM, Xinyi, Jiangsu (CN); Jiyun LONG, Xinyi, Jiangsu (CN); Yizhu HUANG, Xinyi, Jiangsu (CN); Liang ZHANG, Xinyi, Jiangsu (CN)

(21) Appl. No.: 18/007,529

(22) PCT Filed: Apr. 2, 2020
(86) PCT No.: PCT/CN2020/082967

§ 371 Date: Dec. 1, 2022

(51) Int. Cl. A61K 31/23; A61K 9/00; A61P 31/12; A23K 20/105; A23K 20/158

(52) U.S. Cl. CPC A61K 31/23; A61K 9/0056; A61P 31/12; A23K 20/105; A23K 20/158

(57) ABSTRACT

Disclosed is a feed composition, a preparation method therefor, and an application thereof in raising animals. The composition includes a polyglycerol fatty acid ester having a pH value of 2.5-3.5. The fatty acid has 6-14 carbon atoms and is exemplified as laurel acid, caproic acid, caprylic acid, capric acid, or myristic acid. The number of polyglycerol molecules in the polyglycerol fatty acid ester is 1-5.

### FIELD

The present application generally relates to the field of animal feed products and more specifically relates to a composition containing a polyglycerol fatty acid ester (e.g., polyglycerol laurin) applicable to animal feeding, especially the prevention and treatment of animal disease, a preparation method therefor, and application thereof.

### BACKGROUND

In the breeding industry, injections caused by pathogenic microorganisms, such as viruses and bacteria, is a problem that has plagued animal breeding for a long time and is also a challenge and research hotspot that the industry has been facing, and medium- or short-chain fatty acid-based antimicrobial preparations are a research direction. In 1966, John J. Kabara found that lauric acid (C₁₂), a medium-chain fatty acid in saturated fatty acids, has strong antimicrobial activity, while palmitoleic acid (C_{16:1}) in monounsaturated fatty acids has stronger antimicrobial activity. In 2006, Himarsson et al. evaluated the inhibitory effects of monocaprin and monolaurin on the Visna-maedi virus. In 2009, Qingsheng Li found that monolaurin can prevent the mucosal transmission of the simian immunodeficiency virus (SIV). Studies have proven that monolaurin has the strongest inhibitory effect on enveloped viruses. However, monolaurin is typically in the form of a solid, so it cannot achieve a good inhibitory effect on pathogenic microorganisms in vitro when applied in the breeding industry.

The above research direction has good prospects and is also urgently needed in this field.

### SUMMARY

In a first aspect, the present application provides a feed composition, which includes a polyglycerol fatty acid ester and has a pH value of 2.5-3.5. The fatty acid has 6-14 carbon atoms and is exemplified as laurel acid, caproic acid, caprylic acid, capric acid, or myristic acid, and the number of polyglycerol molecules in the polyglycerol fatty acid ester is 1-5.

In some embodiments, the polyglycerol fatty acid ester is polyglycerol laurin. In some embodiments, the polyglycerol laurin is polyglycerol monolaurin.

In some embodiments, the feed composition is in the form of an emulsion. In some embodiments, the emulsion includes one or more of an emulsifier, a stabilizer, an organic acid, and a flavoring agent.

In some embodiments, the emulsifier is selected from citrem, sodium stearoyl lactylate, calcium stearoyl lactylate, mono-/di-glycerides, and combinations thereof.

In some embodiments, the stabilizer is selected from casein, denatured starches (e.g., starch sodium octenyl succinate), carrageenans, and combinations thereof.

In some embodiments, the organic acid is selected from propionic acid, citric acid, lactic acid, and combinations thereof.

In some embodiments, the flavoring agent is selected from sucralose, aspartame, sucrose, and combinations thereof.

In some embodiments, the polyglycerol fatty acid ester is 0.1 wt. %-60 wt. % based on the total weight of the feed composition. In some embodiments, the polyglycerol fatty acid ester is 1 wt. %-30 wt. % based on the total weight of the feed composition. In some embodiments, the polyglycerol fatty acid ester is 10 wt. %-20 wt. % based on the total weight of the feed composition.

In some embodiments, the emulsifier is 0 wt. %-20 wt. % based on the total weight of the feed composition.

In some embodiments, the organic acid is 0.01 wt. %-10 wt. % based on the total weight of the feed composition.

In some embodiments, the flavoring agent is 0 wt. %-5 wt. % based on the total weight of the feed composition.

In some embodiments, the stabilizer is 0 wt. %-40 wt. % based on the total weight of the feed composition.

In a second aspect, the present application provides a preparation method of a feed composition that is in the form of an acidic emulsion including a polyglycerol fatty acid ester and has a pH value of 2.5-3.5. The fatty acid has 6-14 carbon atoms and is exemplified as laurel acid, caproic acid, caprylic acid, capric acid, or myristic acid, and the number of polyglycerol molecules in the polyglycerol fatty acid ester is 1-5. The method includes the following steps:
mixing the polyglycerol fatty acid ester with an emulsifier, a stabilizer, an organic acid, and an optional flavoring agent, regulating a pH value to 2.5-3.5, and emulsifying and homogenizing.

In some embodiments, the polyglycerol fatty acid ester is polyglycerol laurin. In some embodiments, the polyglycerol laurin is polyglycerol monolaurin.

In some embodiments, the emulsifier is selected from citrem, sodium stearoyl lactylate, calcium stearoyl lactylate, mono-/di-glycerides, and combinations thereof.

In some embodiments, the stabilizer is selected from casein, denatured starches (e.g., starch sodium octenyl succinate), carrageenans, and combinations thereof.

In some embodiments, the organic acid is selected from propionic acid, citric acid, lactic acid, and combinations thereof.

In some embodiments, the flavoring agent is selected from sucralose, aspartame, sucrose, and combinations thereof.

In some embodiments, the polyglycerol fatty acid ester is 0.1 wt. %-60 wt. % based on the total weight of the feed composition. In some embodiments, the polyglycerol fatty acid ester is 1 wt. %-30 wt. % based on the total weight of the feed composition. In some embodiments, the polyglycerol fatty acid ester is 10 wt. %-20 wt. % based on the total weight of the feed composition.

In some embodiments, the emulsifier is 0 wt. %-20 wt. % based on the total weight of the feed composition.

In some embodiments, the organic acid is 0.01 wt. %-10 wt. % based on the total weight of the feed composition.

In some embodiments, the flavoring agent is 0 wt. %-5 wt. % based on the total weight of the feed composition.

In some embodiments, the stabilizer is 0 wt. %-40 wt. % based on the total weight of the feed composition.

In a third aspect, the present application provides use of a polyglycerol fatty acid ester in the preparation of a feed composition having a pH value of 2.5-3.5. The fatty acid has 6-14 carbon atoms and is exemplified as laurel acid, caproic acid, caprylic acid, capric acid, or myristic acid, and the number of polyglycerol molecules in the polyglycerol fatty acid ester is 1-5.

In some embodiments, the polyglycerol fatty acid ester is polyglycerol laurin. In some embodiments, the polyglycerol laurin is polyglycerol monolaurin.

In some embodiments, the feed composition is in the form of emulsion. In some embodiments, the emulsion includes one or more of an emulsifier, a stabilizer, an organic acid, and a flavoring agent.

In some embodiments, the emulsifier is selected from citrem, sodium stearoyl lactylate, calcium stearoyl lactylate, mono-/di-glycerides, and combinations thereof.

In some embodiments, the stabilizer is selected from casein, denatured starches (e.g., starch sodium octenyl succinate), carrageenans, and combinations thereof.

In some embodiments, the organic acid is selected from propionic acid, citric acid, lactic acid, and combinations thereof.

In some embodiments, the flavoring agent is selected from sucralose, aspartame, sucrose, and combinations thereof.

In some embodiments, the polyglycerol fatty acid ester is 0.1 wt. %-60 wt. % based on the total weight of the feed composition. In some embodiments, the polyglycerol fatty acid ester is 1 wt. %-30 wt. % based on the total weight of the feed composition. In some embodiments, the polyglycerol fatty acid ester is 10 wt. %-20 wt. % based on the total weight of the feed composition.

In some embodiments, the emulsifier is 0 wt. %-20 wt. % based on the total weight of the feed composition.

In some embodiments, the organic acid is 0.01 wt. %-10 wt. % based on the total weight of the feed composition.

In some embodiments, the flavoring agent is 0 wt. %-5 wt. % based on the total weight of the feed composition.

In some embodiments, the stabilizer is 0 wt. %-40 wt. % based on the total weight of the feed composition.

In some embodiments of the first aspect to the third aspect, the feed composition is used for the prevention or treatment of an enveloped virus infection in an animal such as livestock, poultry, and an aquatic animal. In some embodiments, the enveloped virus is an influenza virus, African swine fever virus (ASFV), porcine reproductive and respiratory syndrome virus, pseudorabies virus or porcine epidemic diarrhea virus.

In a fourth aspect, the present application provides a method for preventing or treating an enveloped virus infection in an animal such as livestock and poultry, which includes the following steps: administering the feed composition described in the first aspect to the third aspect to an animal in need.

### DETAILED DESCRIPTION

The inventors of the present application have conducted an in-depth study on the application of compounds based on fatty acids, especially lauric acid, in resisting animal diseases. As described above, although studies have proven that monolaurin has a strong inhibitory effect on enveloped viruses, it cannot achieve a good inhibitory effect on pathogenic microorganisms in vitro when applied in the breeding industry because it is typically in the form of a solid. Given this, the inventors of the present application polymerize 3 glycerols at the glycine terminus of monolaurin (triglycerol α-monolaurin) to enable that lengths of carbon chains at both sides of the carboxyl terminus are equal. In addition, the inventors have also found that triglycerol α-monolaurin can achieve a better inhibitory effect on enveloped viruses when placed in an acidic emulsion compared to a case where triglycerol α-monolaurin is used alone. Specifically, α-monolaurin is in the form of solid at room temperature, most commercially available products are of 10-40 mesh, and conventional feed materials (e.g., maize) are of 60-80 mesh. Therefore, the chance of contacting enveloped viruses by direction addition of α-monolaurin to feed is very small. Furthermore, α-monolaurin and feed are both particles, there is no liquid medium between the two, even if α-monolaurin can be in contact with viruses, its virus inhibition efficiency is very low and even no. The inventors of the present application modify monolaurin and then prepare the modified monolaurin into liquid polyglycerol laurin with a particle size less than 3 μm, which is more than 100 times smaller than the particle size of the conventional α-monolaurin. Compared with the contact between solid particles, the contact area of liquid polyglycerol laurin with the feed surface is increased by an order of magnitude of 106. In addition, the inventors of the present application have noticed that phosphatidyl polar groups in a lipid bilayer of an enveloped virus attract the modified monolaurin that is also polar to approach and penetrate through the lipid bilayer of the virus, if the length of a carbon chain of hydrophilic groups is equal to that of a carbon chain of hydrophobic groups, and polyglycerol laurin is solidified on the virus envelope or bacterial cell membrane to destroy the lipid membrane of the virus. The inventors of the present application believe that H+ can accelerate this process. Therefore, an inhibitory effect of polyglycerol laurin on viruses is expected to be enhanced by regulating a polyglycerol laurin suspension to be acidic. Based on the above, the inventors of the present application have constructed various inventions of the present application through studies and experiments.

### Definitions

The following definitions are provided to better define the present application and to guide those of ordinary skill in the art in the practice of the present application. Unless otherwise specified, the terms used in the present application have the same meanings as commonly understood by those skilled in the art, such as terms related to raw materials and products, operating steps, process parameters, devices and tools used, and numerical units. All patent documents, academic papers, and other publications cited herein are hereby incorporated by reference in their entireties.

The term “emulsifier” used herein refers to a substance that can improve the surface tension of each constituent phase in an emulsion to enable the constituent phases to form a uniform and stable dispersion system or emulsion. For example, suitable emulsifiers include, but are not limited to, sodium stearoyl lactylate, compound emulsifiers, diacetyl tartaric acid monoglyceride, etc.

The term “compound emulsifier” used herein refers to a combination of one or more emulsifiers. For example, a compound emulsifier may include a glycerol fatty acid ester and a sucrose fatty acid ester as well as sodium-calcium stearoyl lactylate.

The term “stabilizer” used herein refers to a chemical substance that can improve the stability of the fatty acid ester emulsion of the present application. For example, suitable stabilizers include, but are not limited to, casein, denatured starches, carrageenans, etc.

The term “emulsion packaging material” used herein, also referred to as an “emulsion wall material”, refers to other materials rather than emulsifiers that can enable an emulsion to be in a stable state. For example, suitable emulsion packaging materials include, but are not limited to, food colloids, dextrins, syrups, chitosan, lactose, whey powder, etc.

The term “organic acid” used herein refers to some acidic organic compounds that can react with alcohol to form esters. For example, suitable organic acids include, but are not limited to, propionic acid, citric acid, lactic acid, etc.

The term “flavoring agent” used herein refers to an additive that can enable the feed composition of the present application to be more palatable to animals, which is typically a sweetener.

The term “sweetener” used herein refers to an additive that can enable the fatty acid composition emulsion of the present application to be sweet. For example, suitable sweeteners include, but are not limited to, sucralose, aspartame, sucrose, etc.

In a case that there is no apparent conflict, a numerical range described herein is intended to be greater than 0 when it has an endpoint value of 0.

Unless otherwise specified, content percentages (X %) and ratios (X:Y) of ingredients used in the present application are on a basis of weight/weight.

It is to be understood that the term “substantially” or “about” used herein (e.g., component content and a reaction parameter) is interpreted in the meaning commonly understood by those skilled in the art. In general, the term “substantially” or “about” can be understood as any numerical value within plus or minus 5% of a given numerical value. For example, about X or substantially X can represent any numerical value ranging from 95% of X to 105% of X.

It is also understood that specific numerical values given herein (e.g., ratios, temperature, and time) are not only understood as separate numerical values, but also be understood to provide endpoint values for a certain range, and may be combined with each other to provide other ranges.

### DETAILED DESCRIPTION OF THE INVENTION

In a first aspect, the present application provides a feed composition, which includes a polyglycerol fatty acid ester and has a pH value of 2.5-3.5 (e.g., 2.5, 2.6, 2.7, 2.8, 2.9, 3.0, 3.1, 3.2, 3.3, 3.4 or 3.5). The fatty acid has 6-14 (e.g., 6, 7, 8, 9, 10, 11, 12, 13 or 14) carbon atoms and is exemplified as laurel acid, caproic acid, caprylic acid, capric acid, or myristic acid, and the number of polyglycerol molecules in the polyglycerol fatty acid ester is 1-5 (e.g., 1, 2, 3, 4 or 5).

In some embodiments, the polyglycerol fatty acid ester is polyglycerol laurin. In some embodiments, the polyglycerol laurin is polyglycerol monolaurin.

In some embodiments, the feed composition is in the form of emulsion. In some embodiments, the emulsion includes one or more of an emulsifier, a stabilizer, an organic acid, and a flavoring agent.

In some embodiments, the emulsifier is selected from citrem, sodium stearoyl lactylate, calcium stearoyl lactylate, mono-/di-glycerides, and combinations thereof.

In some embodiments, the stabilizer is selected from casein, denatured starches (e.g., starch sodium octenyl succinate), carrageenans, and combinations thereof.

In some embodiments, the organic acid is selected from propionic acid, citric acid, lactic acid, and combinations thereof.

In some embodiments, the flavoring agent is selected from sucralose, aspartame, sucrose, and combinations thereof.

In some embodiments, the polyglycerol fatty acid ester is 0.1 wt. %-60 wt. % (e.g., 0.1, 0.5, 1, 5, 10, 15, 20, 25, 30, 35, 40, 45, 50, 55 or 60 wt. %) based on the total weight of the feed composition. In some embodiments, the polyglycerol fatty acid ester is 1 wt. %-30 wt. % based on the total weight of the feed composition. In some embodiments, the polyglycerol fatty acid ester is 10 wt. %-20 wt. % based on the total weight of the feed composition.

In some embodiments, the emulsifier is 0 wt. %-20 wt. % (e.g., 0.1, 0.5, 1, 5, 10, 15 or 20 wt. %) based on the total weight of the feed composition.

In some embodiments, the organic acid is 0.01 wt. %-10 wt. % (e.g., 0.1, 0.5, 1, 5 or 10 wt. %) based on the total weight of the feed composition.

In some embodiments, the flavoring agent is 0 wt. %-5 wt. % (e.g., 0.1, 0.5, 1 or 5 wt. %) based on the total weight of the feed composition.

In some embodiments, the stabilizer is 0 wt. %-40 wt. % (e.g., 0.1, 0.5, 1, 5, 10, 15, 20, 25, 30, 35 or 40 wt. %) based on the total weight of the feed composition.

As a nonrestrictive example, a formula of the emulsion is shown in the following table.

TABLE 1

| | |
|---|---|
| Polyglycerol monolaurin | 0.1%-60% |
| Citrem | 0%-5% |
| Calcium (sodium) stearoyl lactylate | 0.05%-2% |
| Casein | 0%-8% |
| Mono-/di-glyceride | 0.1%-5% |
| Organic acid | 0.02%-5% |
| Starch sodium octenyl succinate | 0%-25% |
| Water | increase or decrease |
| | according to the balance |

As a more specific nonrestrictive example, a formula of the emulsion is shown in the following table.

TABLE 2

| | |
|---|---|
| Polyglycerol monolaurin | 15% |
| Citrem | 1.3% |
| Calcium (sodium) stearoyl lactylate | 0.7% |
| Casein | 1% |
| Mono-/di-glyceride | 0.8% |
| Organic acid | 2% |
| Starch sodium octenyl succinate | 10% |
| Water | the balance |

In a second aspect, the present application provides a preparation method of a feed composition that is in the form of acidic emulsion including a polyglycerol fatty acid ester and has a pH value of 2.5-3.5. The fatty acid has 6-14 carbon atoms and is exemplified as laurel acid, caproic acid, caprylic acid, capric acid, or myristic acid, and the number of polyglycerol molecules in the polyglycerol fatty acid ester is 1-5. The method includes the following steps:

the polyglycerol fatty acid ester is mixed with an emulsifier, a stabilizer, an organic acid, and an optional flavoring agent, a pH value is regulated to 2.5-3.5, and the mixture is emulsified and homogenized.

In a case that there is no conflict, the technical features described in the first aspect are also applicable to the second aspect.

As a nonrestrictive example, the preparation method is performed as follows.

Polyglycerol monolaurin is heated to about 50-70° C., a usage amount of emulsifier (citrem, calcium (sodium) stearoyl lactylate or a mono-/di-glyceride) is added to and dissolved in the polyglycerol monolaurin, the temperature of the mixture is kept at 50-80° C. for later use as an oil phase; usage amounts of starch sodium octenyl succinate and casein are dissolved in, for example, water, the mixture is heated in a water bath to 80-95° C. until an aqueous solution is formed, the aqueous solution is cooled to 70-80° C. for later use; usage amounts of organic acid and sweetener (optional) are dissolved and added to the mixed solution of starch sodium octenyl succinate and casein, the mixture is stirred until uniform, the temperature of the mixture is kept at 50-80° C. for later use as an aqueous phase; and the oil phase is transferred to the aqueous phase by using a transfer pump, and shear mixing and preliminary emulsification are performed on the mixture. High-pressure homogenization is performed on the mixed solution obtained by preliminary emulsification by using a high-pressure homogenizer under a homogeneous pressure of 15-45 MPa, and the emulsion obtained by high-pressure homogenization is subpackaged.

In a third aspect, the present application provides use of a polyglycerol fatty acid ester in the preparation of a feed composition having a pH value of 2.5-3.5. The fatty acid has 6-14 carbon atoms and is exemplified as laurel acid, caproic acid, caprylic acid, capric acid, or myristic acid, and the number of polyglycerol molecules in the polyglycerol fatty acid ester is 1-5.

In a case that there is no conflict, the technical features described in the first aspect are also applicable to the third aspect.

In some embodiments of the first aspect to the third aspect, the feed composition is used for the prevention or treatment of an enveloped virus infection in an animal such as livestock, poultry, and an aquatic animal. In some embodiments, the enveloped virus is an influenza virus, African swine fever virus (ASFV), porcine reproductive and respiratory syndrome virus, pseudorabies virus or porcine epidemic diarrhea virus.

Animals to which the feed composition of the present application is applicable are not particularly limited, and suitable animals may be, for example, livestock, poultry, and aquatic animals.

The term “livestock” used herein refers to mammals in the breeding industry, such as pigs, cattle, sheep, dogs, cats, and rabbits.

The term “poultry” used herein refers to avian animals in the breeding industry, such as chickens, ducks, geese, and farm birds.

The term “aquatic animal” used herein refers to aquatic animals in the breeding industry, such as fishes, shrimps, crabs, bullfrogs and other farm animals.

An envelope refers to a lipid-like bilayer membrane that is coated on a virus, is composed of proteins, polysaccharides, and lipids, is mainly derived from the host cell membrane (phospholipid layer and membrane protein), also includes some glycoproteins of the virus itself, and mainly helps the virus to enter host cells.

In some embodiments, an enveloped virus is an influenza virus, African swine fever virus (ASFV), porcine reproductive and respiratory syndrome virus, pseudorabies virus or porcine epidemic diarrhea virus

In a fourth aspect, the present application provides a method for preventing or treating an enveloped virus infection in an animal such as livestock and poultry, which includes the following steps: the feed composition described in the first aspect to the third aspect is administered to an animal in need.

### EXAMPLES

The inventions of the present application will be exemplarily described below with reference to examples, but the content of the examples is not intended to limit the inventions of the present application in any manner.

In this example, polyglycerol monolaurin was used as an exemplary polyglycerol fatty acid ester, and its efficacy against an influenza virus infection was tested.

Experimental Materials
Lauric acid was purchased from Guangzhou Easydo Food Co., Ltd.;
monolaurin was purchased from Guangzhou Easydo Food Co., Ltd.;
triglycerol monolaurin was purchased from Guangzhou Easydo Food Co., Ltd.;
a triglycerol monolaurin acidic emulsion was prepared by the inventors of the present application according to the formula in Table 2, the content of triglycerol monolaurin was 15%, and the pH of the emulsion was 2.8;
A549 cells were purchased from Qimeng (Shanghai) Biomedicine Co., Ltd.; and
influenza virus H1N1 was purchased from the Beijing Beina Chuanglian Biotechnology Institute.

Experimental Method

Preparation of Samples

A total of four samples were prepared in this experiment:
(1) a lauric acid sample: 3.5 mg of lauric acid was dissolved in 0.5 mL of dimethyl sulfoxide, and then diluted with 9.5 mL of cell culture medium to a final concentration of 0.35 mg/mL and a pH value of the sample was 10.1;
(2) an α-monolaurin sample: 4.8 mg of α-monolaurin was dissolved in 0.5 mL of dimethyl sulfoxide, and then diluted with 9.5 mL of cell culture medium to a final concentration of 0.48 mg/mL and a pH value of the sample was 10.3;
(3) a triglycerol monolaurin sample: 7.5 mg of triglycerol monolaurin was diluted with 10 mL of cell culture medium to a final concentration of 0.75 mg/mL and a pH value of the sample was 7.6; and
(4) a triglycerol monolaurin acidic emulsion sample: 50 mg of triglycerol monolaurin acidic emulsion sample was diluted with 10 mL of cell culture medium to a final concentration of 5 mg/mL and a pH value of the sample was 7.5.

According to equivalent lauric acid concentration conversion, lauric acid concentrations in the samples (2) to (4) were all 0.35 mg/mL, which was equal to that in the sample (1).

Inoculation of Virus

The A549 cells were cultured in a 24-well plate, after growing to a monolayer, the cells were treated according to the following two protocols:
I. The cell culture medium was removed, the virus was added, the cells were cultured in an incubator with 5% CO₂ at 37° C. for 1 h, the virus solution was removed, each prepared sample was added, 12 parallel test wells were set for each group, and the cells were cultured in the incubator with 5% CO₂ at 37° C. for 1 h. After the culture, liquids in the cell culture wells were removed, the cells were washed with PBS, added with a cell maintenance medium, and cultured in the incubator with 5% CO₂ at 37° C. for 24 h. Meanwhile, a virus positive control group (a cell culture medium mixed with influenza virus 24HAU was added) was set.
II. The cell culture medium was removed, each prepared sample and the virus were added, 12 parallel test wells were set for each group, and the cells were cultured in an incubator with 5% CO₂ at 37° C. for 1 h. After the culture, liquids in the cell culture wells were removed, the cells were washed with PBS, added with a cell maintenance medium, and cultured in the incubator with 5% CO₂ at 37° C. for 24 h. Meanwhile, a virus positive control group (a cell culture medium mixed with influenza virus 24HAU was added) was set.

Cell culture supernatants respectively obtained by the above 2 treatment methods were used for hemagglutination test, each culture plate was frozen and thawed repeatedly 3 times, a cell solution was collected for extraction of RNA, which was used for qRT-PCR detection of nucleoprotein (NP).

Experimental Results

Results of Hemagglutination Test

TABLE 3

| Inoculation | | | | | Virus |
|---|---|---|---|---|---|
| treatment | (1) | (2) | (3) | (4) | control |
| Protocol 1 | 4, 5 | 2, 3 | 4, 4 | 3, 4 | 8, 9 |
| Protocol 2 | 5, 5 | 2, 2 | 4, 5 | 2, 3 | |
| Average | 4.75 | 2.25 | 4.25 | 3 | 8.5 |
| values | | | | | |

It can be seen from the results in Table 3 that averages hemagglutination titers of the two treatment methods from low to high are: (2), (4), (3), and (1). High hemagglutination titer indicates high virus content, and it can thus be seen that the inhibitory effect of the sample (2) (α-monolaurin) on the influenza virus is the strongest, followed by the sample (4) (triglycerol monolaurin acidic emulsion), and the effects of the samples (1) and (3) are weaker compared to the sample (4).

qRT-PCR detection results of nucleoprotein (NP) have the same trend as the above hemagglutination test results, and virus contents from low to high are: (2), (4), (3), and

The hemagglutination test results and the qRT-PCR detection results of nucleoprotein all show that the acidic emulsion of the present application has a strong inhibitory effect on influenza virus H1N1, which is slightly weaker than the known inhibitory effect of monolaurin on enveloped viruses, but monolaurin is difficult to apply to the breeding industry due to the limitation of its physical properties. Furthermore, it can be seen through comparison of the results of the samples (3) and (4) that an antiviral effect of triglycerol monolaurin will be greatly enhanced after triglycerol monolaurin is prepared into an acidic emulsion.

The present application has been described in detail above with general description and specific embodiments, but some modifications or improvements may be made to the present application, which will be obvious to those skilled in the art. Therefore, these modifications or improvements made without departing from the spirit of the present application shall fall within the scope of protection claimed by the present application.

1.-15. (canceled)

16. A feed composition comprising a polyglycerol fatty acid ester and having a pH value of 2.5-3.5, wherein the polyglycerol fatty acid ester comprises a C₆-C₁₄ fatty acid moiety, and a number of polyglycerol moieties in the polyglycerol fatty acid ester is 1-5.

17. The feed composition according to claim 16, wherein a fatty acid of the polyglycerol fatty acid ester is at least one selected from a group consisting of laurel acid, caproic acid, caprylic acid, and myristic acid.

18. The feed composition according to claim 16, wherein the polyglycerol fatty acid ester is polyglycerol laurin.

19. The feed composition according to claim 16, wherein the polyglycerol fatty acid ester is polyglycerol monolaurin.

20. The feed composition according to claim 16, wherein the feed composition is in a form of an emulsion.

21. The feed composition according to claim 20, wherein the feed composition comprises at least one selected from a group consisting of an emulsifier, a stabilizer, an organic acid, and a flavoring agent.

22. The feed composition according to claim 21, wherein at least one of:
the emulsifier is at least one selected from a group consisting of citrem, sodium stearoyl lactylate, calcium stearoyl lactylate, and mono-/di-glycerides;
the stabilizer is at least one selected from a group consisting of casein, denatured starch, and carrageenan;
the organic acid is at least one selected from a group consisting of propionic acid, citric acid, and lactic acid; or
the flavoring agent is at least one selected from a group consisting of sucralose, aspartame, and sucrose.

23. The feed composition according to claim 22, wherein the denatured starch is starch sodium octenyl succinate.

24. The feed composition according to claim 21, wherein, based on a total weight of the feed composition, at least one of:
the polyglycerol fatty acid ester is 0.1 wt. %-60 wt. %;
the emulsifier is 0 wt. %-20 wt. %;
the organic acid is 0.01 wt. %-10 wt. %;
the flavoring agent is 0 wt. %-5 wt. %; or
the stabilizer is 0 wt. %-40 wt. %.

25. The feed composition according to claim 21, wherein, based on a total weight of the feed composition, the polyglycerol fatty acid ester is 1 wt. %-30 wt. %.

26. The feed composition according to claim 21, wherein, based on a total weight of the feed composition, the polyglycerol fatty acid ester is 10 wt. %-20 wt. %.

27. A preparation method of the feed composition according to claim 16, comprising the following steps:
mixing the polyglycerol fatty acid ester with an emulsifier, a stabilizer, and an organic acid, regulating the pH value to 2.5-3.5, and emulsifying and homogenizing.

28. A preparation method of the feed composition according to claim 16, comprising the following steps:
mixing the polyglycerol fatty acid ester with an emulsifier, a stabilizer, an organic acid, and a flavoring agent, regulating the pH value to 2.5-3.5, and emulsifying and homogenizing.

29. A method of preventing or treating an enveloped virus infection in an animal comprising administrating the feed composition of claim 16.

30. The method according to claim 29, wherein the animal is livestock, poultry, or an aquatic animal.

31. The method according to claim 29, wherein an enveloped virus corresponding to the enveloped virus infection is selected from influenza viruses, African swine fever virus (ASFV), porcine reproductive and respiratory syndrome virus, pseudorabies virus, and porcine epidemic diarrhea virus.
